(19) United States
(12) Patent Application Publication
(10) Pub. No.: US 2016/0303288 A1
(43) Pub. Date: Oct. 20, 2016

(54) METHOD FOR PREPARING BIOCOMPATIBLE CORNEA AND DECELLULARIZATION COMPOSITION FOR BIOCOMPATIBLE TISSUE

(71) Applicant: POSTECH ACADEMY-INDUSTRY FOUNDATION, Gyeongsangbuk-do (KR)

(72) Inventors: Moon Nyeo Park, Gyeongsangbuk-do (KR); Dong Woo Cho, Seoul (KR); Young Mi Moon, Gyeongsangbuk-do (KR); Jinah Jang, Gyeongsangbuk-do (KR); Hyeon Ji Kim, Gyeongsangbuk-do (KR)

(21) Appl. No.: 15/131,302

(22) Filed: Apr. 18, 2016

(30) Foreign Application Priority Data
Apr. 17, 2015 (KR) 10-2015-0054725

(51) Int. Cl. A61L 27/36; B33Y 80/00; B33Y 70/00; B33Y 10/00

(52) U.S. Cl. CPC A61L 27/3687; A61L 27/3604; B33Y 10/00; B33Y 80/00; B33Y 70/00; A61L 2430/16

(57) ABSTRACT

Disclosed is a method for preparing a biocompatible tissue, the method including: providing a cornea from a tissue source through corneal incision; decellularizing the provided cornea in purified water containing charcoal for a predetermined period of time; and post-treating the decellularized corneal extracellular matrix through stirring in a hypotonic solution, so that the charcoal is used as a decellularizing agent for the cornea, thereby allowing the regeneration of a corneal tissue like in the original corneal extracellular matrix, and the preparation of a cornea without an immune rejection response and a fast regeneration effect of a patient through transplantation of the cornea can be expected.

### CROSS-REFERENCE TO RELATED APPLICATIONS

This application claims the benefit of priority of Korean Patent Application No. 10-2015-0054725 filed on Apr. 17, 2015, all of which are incorporated by reference in their entirety herein.

### BACKGROUND OF THE INVENTION

1. Field of the invention

The present invention relates to a method for preparing a biocompatible cornea and a decellularization composition for a biocompatible tissue and, more specifically, to a method for preparing a biocompatible cornea by decellularizing biological tissues using biocompatible charcoal and to a decellularization composition for a biocompatible tissue.

2. Related Art

The cornea is the transparent membrane that covers the surface of the black pupil. The cornea performs not only a primary role of protecting eyes from the outside, but also a window role of receiving light in the eye to allow things to be seen.

A structurally or functionally irreparable damage on the cornea causes corneal edema or corneal opacity, resulting in bad eyesight. Moreover, extremely opaque cornea may cause beauty problems. Therefore, a corneal transplant in that the damaged cornea is exchanged with a clean cornea donated by other person is widely employed.

However, the corneas donated for cornea transplants are extremely insufficient, and complications, such as infections and immune responses, may be developed after the cornea transplant. Recently, a technique has been studied that use a 3D printer to prepare artificial corneal tissues suitable for patients. However, biodegradable polymers used to prepare artificial corneas have low cell adhesion and less differentiation into corneal tissues.

### SUMMARY OF THE INVENTION

In tissue engineering, the decellularized extracellular matrix is variously applied, and may be used as one of the most suitable bio-derived materials. An aspect of the present invention is that charcoal, which is a nature-friendly material, is used as a decellularizing agent, rather than chemical treatment, for the effective use of the decellularized extracellular matrix.

An aspect of the present invention is to induce the promotion of corneal tissue regeneration by performing decellularization such that a regular arrangement of collagen and glycosaminoglycans (GAGs), constituting the extracellular matrix of the corneal tissue, is not damaged.

In an aspect, a method for preparing a biocompatible corneal tissue is provided. The method includes: providing a cornea from a tissue source; decellularizing the provided cornea in purified water containing charcoal for a predetermined period of time; and post-treating the decellularized corneal extracellular matrix through stirring in a hypotonic solution.

Furthermore, in the decellularizing step, negative ions emitted from the charcoal contained in the purified water may stabilize negative charges on a surface of collagen or glycosaminoglycans (GAGs) constituting the extracellular matrix of the provided cornea.

Furthermore, in the decellularizing step, the charcoal contained in the purified water may may be porous, and may adsorb or remove impurities in the periphery of the provided cornea tissue through pores thereof.

Furthermore, in the decellularizing step, pulverized charcoal and a salt may be placed in purified distilled water, and then the provided cornea may be placed therein, followed by stirring at 150-210 rpm for 18-20 hours.

Furthermore, the content of DNA, which is contained in the corneal extracellular matrix decellularized with the charcoal contained in the purified water, may be less than 3% by mass.

Furthermore, in the post-treating step, the decellularized extracellular matrix may be placed in the hypotonic solution, followed by stirring at 150-210 rpm for 18-30 hours, while Tris-HCl (pH 8) is used as the hypotonic solution.

Furthermore, the method may further include: after the post-treating step, washing the post-treated corneal extracellular matrix with a buffer and purified distilled water, followed by lyophilization for a predetermined period of time; incising the lyophilized corneal extracellular matrix, followed by solubilization; and adjusting the solubilized matrix to the pH suitable for the human body, and then inducing gelation of the matrix.

Furthermore, in the solubilizing step, the corneal extracellular matrix may be treated with one selected from the group consisting of protein hydrolases, corresponding to 0.01-0.05 mass percent of the corneal extracellular matrix, followed by addition of an acid treatment material and then stirring at 500-700 rpm for 44-52 hours.

Furthermore, the method may further include: after the step of inducing gelation, injecting the gelated bioink into a 3D printer, and preparing a cornea matching the size of a scanned affected part of a transplant recipient using the 3D printer; transplanting the prepared cornea into the transplant recipient; and allowing the transplanted cornea to grow in contact with cells in the periphery of the cornea of the transplant recipient.

In another aspect, a decellularization composition for a biocompatible tissue is provided. The decellularization composition is a mixture for decellularization of a tissue provided from a tissue source and contains purified water and charcoal as active ingredients.

Furthermore, negative ions emitted from the charcoal may stabilize negative charges on a surface of collagen or glycosaminoglycans (GAGs) constituting the extracellular matrix of the provided cornea.

Furthermore, the charcoal may be porous, and may adsorb or remove impurities in the periphery of the provided cornea through pores thereof.

Furthermore, the mixture may contain pulverized charcoal and a salt in the purified water.

The present invention has an excellent effect of regenerating the corneal tissue like in the original corneal extracellular matrix, and can minimize the immune rejection response.

### BRIEF DESCRIPTION OF THE DRAWINGS

FIG. 1 is a flowchart showing a method for preparing a biocompatible cornea according to an embodiment of the present invention.

FIG. 2 is an analysis graph from a test on whether the corneal extracellular matrix decellularized with charcoal according to an embodiment of the present invention is adequate as a biomaterial.

FIG. 3 is an analysis graph from a test on whether the cartilage extracellular matrix decellularized with charcoal according to an embodiment of the present invention is adequate as a biomaterial.

FIG. 4 is a graph comparing cell viability through treatment with CCK-8 according to an embodiment of the present invention.

FIG. 5 illustrates images showing cell viability in the matrix decellularized with charcoal.

### DESCRIPTION OF EXEMPLARY EMBODIMENTS

Hereinafter, the method for preparing a biocompatible cornea according to an embodiment of the present invention will be described in detail with reference to the accompanying drawings. However, the terms or words disclosed herein are not delimited to only conventional or dictionary meanings, and should be construed to have meanings and concepts that coincide with the technical scope of the present invention on the basis of the principle that the concepts of the terms can be properly defined in order to illustrate the invention of the inventor.

Therefore, the features described in embodiments and shown in drawings in the present specification are for merely illustrating the most preferable embodiments, but are not intended to represent the technical spirit of the present invention, and thus the present invention may cover various equivalents and modifications which can substitute for the embodiments at the time of filing the present application.

Furthermore, the method for preparing a biocompatible cornea and the decellularization composition for a biocompatible cornea according to the present invention are described by limiting to the cornea, but the present invention are not limited thereto, and thus the present invention can be applied to various body tissues in addition to the cornea.

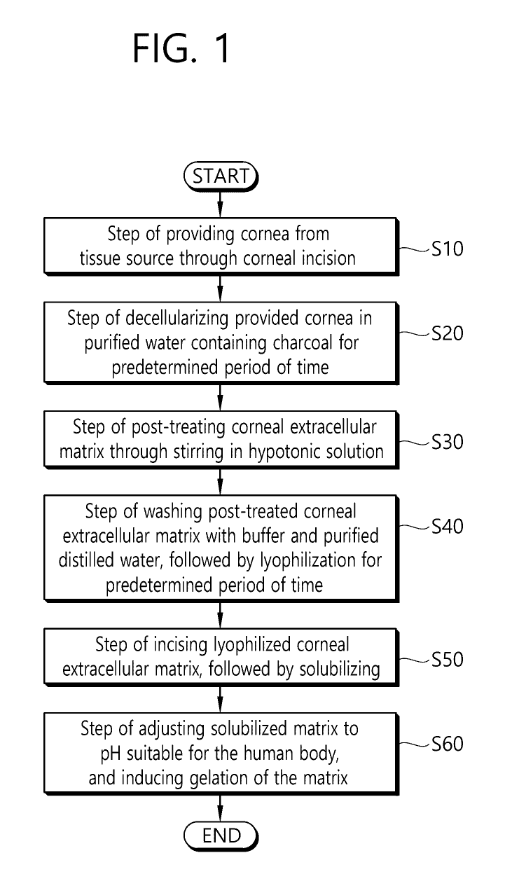

FIG. 1 is a flowchart showing a method for preparing a biocompatible cornea according to an embodiment of the present invention.

First, a step is carried out that provides a cornea from a tissue source through corneal incision (S10).

The tissue source may be selected from the group of various animals, such as pigs, cows, and rabbits. In the present invention, a bovine cornea was used as a tissue source. Therefore, the bovine cornea may be incised in a pupil shape with 5-13 mm. In addition, the epithelium is removed and the endothelium is peeled.

Thereafter, 450-550 um-thick stromal layers with the epithelium and endothelium removed may be placed in a buffer, and then may be washed through stirring at 140-210 rpm for 30 minutes to 1 hour. Here, phosphate buffer saline (PBS) may be used as the buffer. Thereafter, the washing-completed corneal extracellular matrix is freeze-stored for 12-20 hours. The gap between the stroma layers may be widened through the freeze storage.

In addition, a step may be carried out that decellularizes the provided cornea in purified water containing charcoal for a predetermined period of time (S20).

Specifically, in the decellularizing step, first, the stroma layers inflated through the freeze storage may be peeled into two to four layers along the grain. However, since the stroma layers have different thicknesses depending on the kind of tissue source and the growth thereof, the number of peeled stroma layers may be selectively varied without the limitation to two to four layers.

Thereafter, the frozen cornea may be placed in a solution in which charcoal and a salt are contained in previously prepared DI water, followed by stirring at 150-210 rpm for 20-28 hours. Here, the charcoal may be added in the solution while precisely pulverized charcoal is placed in a tea bag, and NaCl may be used as the salt. Here, the charcoal may function as a decellularizing agent.

Charcoal has many effects of purification, cleaning, deodorization, detoxification, refinement, removal of electromagnetic waves, and the like. Of these, the emission of far infrared radiation and negative ions is one of the strongest functions of charcoal that directly influence out health. Here, the emission of negative ions from the charcoal can stabilize the structure of negative charges on the surfaces of collagen and glycosaminoglycans (GAGs) constituting the extracellular matrix (ECM).

Collagen is the main protein in all connective tissues, such as skin, blood vessels, bones, teeth, and muscles, and is also present as the intercellular matrix in other organs. Collagen is composed of three polypeptide chains twisting into a triple helix structure, and has a high content of hydroxyproline, and especially, collagen, in the form of elongated fibrils, constituting mammal flesh and connective tissues may mainly act as a main component for tendons, ligaments, corneas, cartilage tissues, and the like.

Glycosaminoglycans (GAGs) are present in the periphery of proteoglycan in the extracellular matrix (ECM) and on the cell surfaces, and are long polysaccharides having several disaccharides. The proteoglycan near to glycosaminoglycans (GAGs) binds to growth factors and cytokines in the extracellular matrix (ECM), and thus may function to directly provide environments for differentiation or proliferation to the cells.

The corneal tissue, compared with other tissues, maintains the eye sight through a regular arrangement of collagen and glycosaminoglycans (GAGs) constituting the extracellular matrix (ECM). That is, collagen and glycosaminoglycans (GAGs) account for most of the corneal tissue, compared with other tissues, and the percents of the collagen and the glycosaminoglycans (GAGs) and the arrangement thereof are important factors in retaining distinctive characteristics of the cornea.

In the present embodiment, charcoal is used as a decellularizing agent in the decellularizing procedure, and here, the characteristics of the charcoal emitting negative ions stabilize the surface negative charge structure of collagen and glycosaminoglycans (GAGs) constituting the extracellular matrix of the cornea, thereby maintaining a regular arrangement of collagen or glycosaminoglycans (GAGs).

Most detergents, such as CHAPS, used as an existing decellularizing agent, deforms the surface charge of collagen and glycosaminoglycans (GAGs), causing the structure deformation and damage of the tissue, and thus were disadvantageous in the tissue regeneration. In the present invention, charcoal, as a decellularizing agent, has a function of maintaining an arrangement of collagen and glycosaminoglycans (GAGs), and thus favorably acts in the preservation of the extracellular matrix (ECM), thereby preparing a tissue with excellent biocompatibility.

In addition, charcoal is porous, and pores contained in the charcoal can adsorb impurities corresponding to microorganisms, heavy metals, and pollutants. The charcoal having these pores adsorbs or removes the impurities in the periphery of the extracellular matrix (ECM) of the provided cornea, thereby effectively removing the materials interfering with the tissue regeneration.

After the decellularizing step using charcoal, a step may be carried out that post-treats the corneal extracellular matrix, decellularized in the foregoing step, in a hypotonic solution through stirring (S30).

In the post-treating step, Tris-HCl (pH 8) is used as the hypotonic solution, and the decellularized cornea is placed in the hypotonic solution, followed by stirring at 150-210 rpm for 18-30 hours. Through the post-treating step above, DNA or growth factors remaining after cell lysis may be removed.

High contents of DNA or growth factors in the decellularized extracellular matrix (ECM) cause an immune response due to the recognition of autologous cells. Therefore, most of DNA or growth factors need to be removed through the post-treating step. As described later, the present invention has an advantage of obtaining the extracellular matrix containing a lower content of DNA through the decellularizing step using charcoal.

Thereafter, a step may be carried out that washes the post-treated corneal extracellular matrix with a buffer and purified distilled water, followed by lyophilization for a predetermined period of time (S40).

The washing step is carried out through several stages, and the impurities are sufficiently removed through the washing, thereby minimally suppressing the immune response when the extracellular matrix is transplanted in the tissue.

In the washing step, filtered PBS is used as a buffer, and the washing may be carried out with filtered PBS and purified DI water through stirring at 150-210 rpm for 24-48 hours. Thereafter, the stirring may be carried out for 20-28 hours while using 8-12 mM Tris buffer (pH 8) as an additional buffer. In addition, 1% triton and 10 mM Tris buffer are placed, followed by stirring for 20-40 minutes. Thereafter, the stirring may be carried out in 0.1% peracetic acid, 5% ethanol, and 94.9% DI water at 150-210 rpm for about 1 hour. In addition, lastly, the washing may be completed through stirring for 12-20 hours and stirring using PBS for 24-48 hours. A step may be carried out that freeze-stores the decellularized corneal extracellular matrix (ECM) on the completion of the washing, followed by lyophilization.

After, a step may be carried out that incises the lyophilized corneal extracellular matrix (ECM), followed by solubilizing (S50).

The sufficiently lyophilized cornea needs to be finely pulverized to be suitable for the progress of the gelation step. Therefore, the lyophilized decellularized corneal extracellular matrix may be made into fine particles through fine cutting with scissors or blending with a mixer. Thereafter, the finely pulverized extracellular matrix particles are subjected to a solubilization procedure. Specifically, the corneal extracellular matrix particles may be treated with one selected from the group consisting of protein hydrolases, corresponding to 0.01-0.05 by mass of the corneal extracellular matrix, followed by addition of an acid treatment material and stirring at 500-700 rpm for 44-52 hours. Herein, pepsin was used as the protein hydrolase, and about 0.5 M acetic acid was used as the acid treatment material.

A step may be carried out that, on the completion of solubilization, adjusts the particulated corneal extracellular matrix (ECM) to the pH suitable for the human body, and induces gelation of the matrix (S60).

Specifically, the pH is adjusted to 7.2-7.6 by adding 10 M NaOH of about 10 ul for each time. After the pH is properly adjusted, the extracellular matrix (ECM) is placed in an incubator, and then the gelation is induced. Therefore, the solution subjected to the gelation procedure may be used as a raw material for a corneal regeneration member using a 3D printer.

The gelated extracellular matrix is a bioink, which is injected into the 3D printer, to prepare a corneal graft that accurately matches an affected part of a patient.

The 3D printer, first, scans the size of the affected part of a transplant recipient, thereby securing corneal data matching the size of the affected part of the transplant recipient. Thereafter, a step may be carried out that injects a viscous solution composed of the gelated decellularized extracellular matrix, that is, the bioink into a 3D printer and prepares a cornea matching the size of the affected part of the transplant recipient, which is scanned using the 3D printer.

After that, the transplantation is completed by going through a step of transplanting the prepared cornea into the transplant recipient and a step of allowing the transplanted cornea to grow in contact with cells in the periphery of the cornea of the transplant recipient.

The extracellular matrix decellularized with charcoal allows an effective removal of impurities therefrom due to the foregoing efficacies of charcoal and a favorable fusion with cells in the periphery of the transplant tissue of the transplant recipient due to excellent biocompatibility thereof. In addition, the differentiation of stem cells favorably occurs, leading to fast and effective corneal transplantation, and thus, a fast regeneration effect of the patient receiving the cornea can be expected.

Furthermore, the present invention, in order to effectively apply the utility of the decellularized extracellular matrix, employed, as a decellularizing agent, charcoal, which is a nature-friendly material, instead of employing chemical treatment. The charcoal used in the decellularized extracellular matrix helps the world better understand about the excellence of materials that are traditionally used in Korea and can be utilized as a good material in tissue engineering for global targeting.

Hereinafter, in order to whether the corneal extracellular matrix (ECM) decellularized with charcoal is adequate as a biomaterial, the change in percents of favorable components constituting the cornea and the gene removal effect were analyzed through tests.

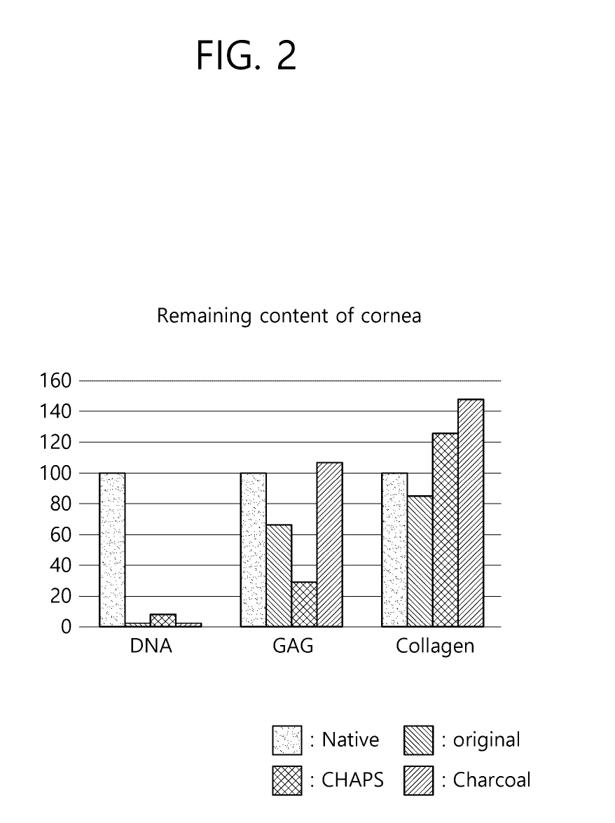

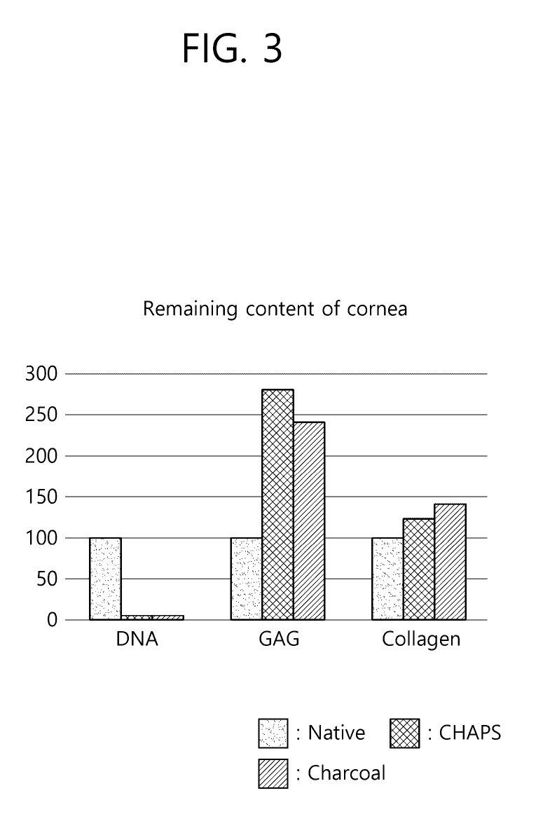

FIG. 2 is an analysis graph from a test on whether the corneal extracellular matrix decellularized with charcoal according to an embodiment of the present invention is adequate as a biomaterial. FIG. 3 is an analysis graph from a test on whether the cartilage extracellular matrix decellularized with charcoal according to an embodiment of the present invention is adequate as a biomaterial.

Referring to FIGS. 2 and 3, the hydroxyproline assay for quantification of total collagen content was conducted for the decellularization analysis of the biomaterial.

Here, hydroxyproline is an enzyme that synthesizes collagen. First, a standard solution (30 ug/ml) is diluted to 0, 1, 2, 3, 4, 5, 6, 8, 10, 15, 20, and 30 ug/ml. In addition, three samples for each were placed in a 96-well plate. In addition, a chloramine T solution was added, and then left at room temperature (RT) for 20 minutes. Additionally, 3,3′-diaminobenzidine (DAB) was added, and then left in a water bath at a temperature of 60° C. for 30 minutes. After that, the mixture was cooled at room temperature (RT), and the quantity of collagen was analyzed through the absorbance at 540 nm wavelength.

In addition, the quantification of DNA using Hoechst 33258 was conducted. The quantification of DNA using Hoechst 33258 is an analysis method of quantifying template DNA synthesized by DNA polymerase isolated from the thymus.

First, the calf thymus DNA standard stock solution (1 mg/ml) is diluted to 1000, 500, 250, 100, 50, 20, 10, and 0 ng/ml using TE buffer. Three samples for each are prepared by dilution to 1/10. Here, if each sample is measured to have 3% or more DNA, an immune response occurs against external tissues due to the recognition of autologous cells, and thus the use of such a decellularized extracellular matrix is not adequate. However, it was verified that the corneal or cartilage extracellular matrix decellularized with charcoal showed a more favorable effect, compared with CHAPS used as an existing decellularizing agent.

In addition, dimethylmethylene blue assay (DMMB) used in the test was further conducted. The dimethylmethylene blue (DMMB) is a positive ion dye, and binds to only sulfated glycosaminoglycan. In other words, DMMB binds to only chondroitin sulfate, keratin sulfate, and heparan sulfate, and thus DMMB is used to quantify GAGs.

20 ug/ml CSA (a), 40 ug/ml CSA (b), and 60 ug/ml CSA (c) are prepared, and three samples are prepared for each. DMMA dye is placed in each well, and the absorbance value at 535 nm wavelength is read. The quantity was measured through the comparison between the standard value and the sample value.

Table 1 shows comparision of contents of DNA, GAG, and collagen in the corneal extracellular matrix decellularized with CHAPS or charcoal through the above test.

TABLE 1
Cornea

| | DNA | GAG | Collagen |
|---|---|---|---|
| Native | 100 | 100 | 100 |
| Original | 2.34 | 67.00 | 85.15 |
| CHAPS | 8.26 | 29.37 | 125.82 |
| Charcoal | 2.89 | 107.18 | 148.09 |

Native represents the original corneal tissue; Original represents the decellularized corneal tissue; CHAPS represents the extracellular tissue treated with CHAPS; and Charcoal represents the extracellular tissue treated with charcoal.

It can be seen from FIG. 2 and table 1 that the corneal extracellular matrix treated with charcoal had a lower DNA content and higher contents of glycosaminoglycans (GAGs) and collagen than the extracellular matrix treated with CHAPS used as an existing decellularizing agent. As described above, in order to suppress the immune response, a lower DNA content leads to a more stable value, and a DNA content of less than 3% is suitable. In addition, the eye sight is maintained according to an arrangement of collagen and glycosaminoglycans (GAGs) contained in the extracellular matrix (ECM), and thus higher contents of glycosaminoglycans (GAGs) and collagen are advantageous.

Therefore, as for the corneal extracellular matrix (ECM) treated with charcoal, the content of DNA on the basis of the total weight is less than 3%, which is stable, and the contents of glycosaminoglycans (GAGs) and collagen are very high compared with the extracellular matrix (ECM) using CHAPS, and therefore, the corneal extracellular matrix (ECM) treated with charcoal exhibits more favorable effects in view of corneal formation and biocompatibility.

Furthermore, charcoal corresponds to an economically favorable material compared with CHAPS. For example, currently, charcoal forms a price range of approximately 30,000 Korean won per 5 kg, whereas CHAPS forms a price range of 170,000 Korean won per 120 ml. Therefore, charcoal has a very favorable advantage in view of economical efficiency as well as direct effects as a decellularizing agent for the cornea.

Table 2 shows comparision of contents of DNA, GAG, and collagen in the cartilage extracellular matrix decellularized with CHAPS or charcoal through the above test.

TABLE 2
Cartiliage

| | DNA | GAG | Collagen |
|---|---|---|---|
| Native | 100 | 100 | 100 |
| CHAPS | 4.67 | 279.80 | 122.94 |
| Charcoal | 4.70 | 240.30 | 141.52 |

Native represents the original cartilage tissue; CHAPS represents the cartilage extracellular tissue treated with CHAPS; and Charcoal represents the cartilage extracellular tissue treated with charcoal.

It can be seen from FIG. 3 and table 2 that the cartilage extracellular matrix decellularized with charcoal had a similar DNA content and higher contents of glycosaminoglycans (GAGs) and collagen than the extracellular matrix treated with CHAPS used as an existing decellularizing agent. As described above, in order to suppress an immune response, a lower DNA content leads to a more stable value, and in order to regenerate the cartilage using collagen and glycosaminoglycans (GAGs) constituting most of cartilage, higher contents of collagen and glycosaminoglycans (GAGs) are advantageous. Meanwhile, as for cartilage, it is known that the stable DNA content in the extracellular matrix (ECM) is generally within 5%.

Therefore, the DNA content on the basis of the total weight in the cartilage extracellular matrix decellularized with charcoal is not greatly different from that in the extracellular matrix using CHAPS, but shows a stable value, and the contents of collagen and glycosaminoglycans (GAGs) in the cartilage extracellular matrix decellularized with charcoal are much higher compared with those in the extracellular matrix using CHAPS, indicating a more favorable effect in the formation of the cartilage tissue. These results could again confirm that the matrix decellularized with charcoal can contain high contents of components necessary for the tissue regeneration in the corneal tissue and other tissues.

In conclusion, the corneal extracellular matrix (ECM) decellularized with charcoal has a low content of DNA, which causes an immune response, and high contents of collagen and glycosaminoglycans (GAGs), which are the most important components in the tissue regeneration, and thus the corneal extracellular matrix (ECM) decellularized with charcoal can have an excellent effect as a transplant material and can produce economical efficiency.

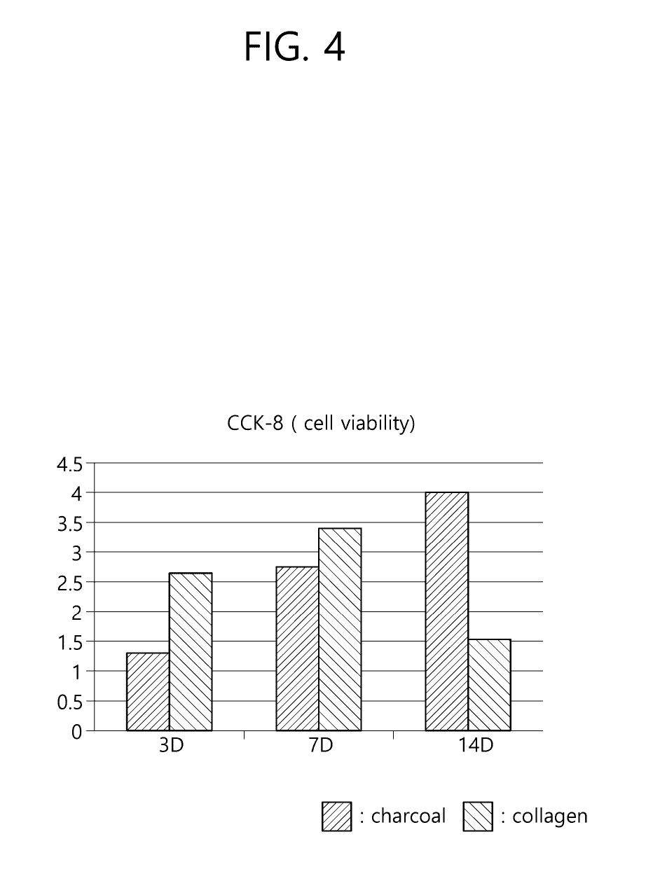

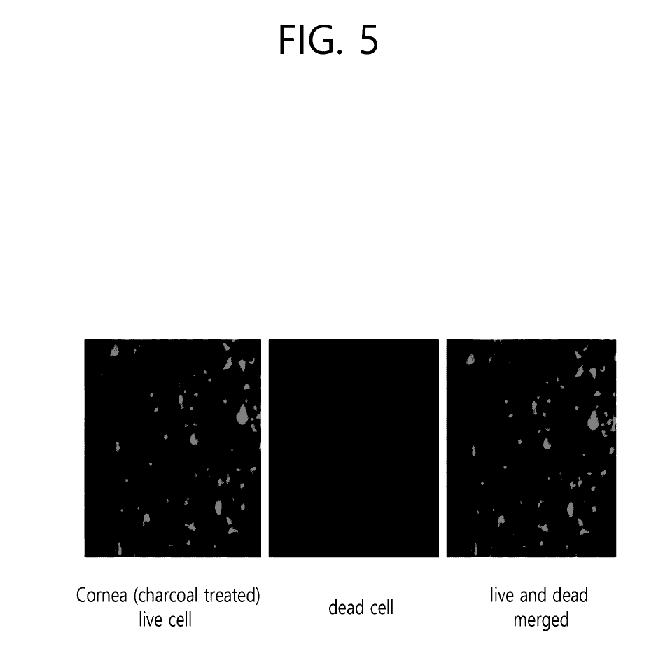

FIG. 4 is a graph showing the comparision of cell viability through treatment with CCK-8 according to an embodiment of the present invention. FIG. 5 illustrates images showing cell viability in the matrix decellularized with charcoal.

Referring to FIG. 4, in order to investigate cell viability, CCK-8 that is generally used was employed as a chemical. On the graph, bars indicated by “charcoal” represent cell viability over time after cells are placed in the extracellular matrix decellularized with charcoal. Bars indicated by “collagen” represent cell viability over time after cells are placed in collagen. Comparison was conducted at days 3, 7, and 14.

Over time, it could be confirmed that the cell viability continuously increased in the group in which cells were placed in the decellularized extracellular matrix rather than in the group in which cells were placed in collagen.

Referring to FIG. 5, the distribution of live cells was compared, through the images, with the distribution of dead cells in the cornea, which was subjected to a decellularizing process using charcoal, and the cell viability was confirmed to be active.

Therefore, it could be seen from FIGS. 4 and 5 that the use of charcoal as a decellularizing agent showed a fast tissue regeneration effect and more favorable effects in corneal formation and biocompatibility.

The decellularization composition for a biocompatible tissue according to the present invention may contain, as active ingredient, purified water and charcoal in a mixture for decellularization of a tissue provided from a tissue source. The tissue may contain a cornea, and hereinafter, the present invention will be described by a cornea as an example. In addition, the mixture may contain pulverized charcoal and a salt in the purified water. Here, the charcoal may perform as a decellularizing agent in a manner in which the charcoal is thin pulverized and placed in a tea bag, so that the charcoal is contained in the solution. In the present invention, NaCl may be used as the salt.

Therefore, the decellularization may be conducted by placing the frozen cornea in the decellularization composition composed of the active ingredients, followed by stirring at 150-210 rpm for 20-28 hours.

As described above, the charcoal contained in the decellularization composition may emit negative ions. That is, the negative ions emitted from the charcoal can stabilize negative charges on the surface of collagen or glycosaminoglycans (GAGs) constituting the provided corneal extracellular matrix. Besides, the charcoal is porous, and has an effect of adsorbing or removing impurities in the periphery of the provided cornea tissue, through pores of the charcoal. Since the effects of charcoal used as a decellularizing agent and specific methods thereof have been described above, the descriptions thereof are omitted.

Although the embodiments of the present invention has been described with reference to the accompanying drawings, a person skilled in the art to which the present invention pertains should apprehend that the present invention can be embodied in other specific forms without departing from the technical spirit or essential characteristics thereof. Therefore, the embodiments described above should be construed as being exemplified and not limiting the present disclosure. The scope of the present invention is not defined by the detailed description as set forth above but by the accompanying claims of the invention, and it should also be understood that all changes or modifications derived from the definitions and scopes of the claims and their equivalents fall within the scope of the invention.

What is claimed is:

1. A method for preparing a biocompatible corneal tissue, the method comprising:
providing a cornea from a tissue source;
decellularizing the provided cornea in purified water containing charcoal for a predetermined period of time; and
post-treating the decellularized corneal extracellular matrix through stirring in a hypotonic solution.

2. The method of claim 1, wherein in the decellularizing step, negative ions emitted from the charcoal contained in the purified water stabilize negative charges on a surface of collagen or glycosaminoglycans (GAGs) constituting the extracellular matrix of the provided cornea.

3. The method of claim 1, wherein in the decellularizing step, the charcoal contained in the purified water is porous, and adsorbs or removes impurities in the periphery of the provided cornea tissue through pores thereof.

4. The method of claim 1, wherein in the decellularizing step, pulverized charcoal and a salt are placed in purified distilled water, and then the provided cornea is placed therein, followed by stirring at 150-210 rpm for 18-20 hours.

5. The method of claim 1, wherein the content of DNA, which is contained in the corneal extracellular matrix decellularized with the charcoal contained in the purified water, is less than 3% by mass.

6. The method of claim 1, wherein in the post-treating step, the decellularized extracellular matrix is placed in the hypotonic solution, followed by stirring at 150-210 rpm for 18-30 hours, while Tris-HCl (pH 8) is used as the hypotonic solution.

7. The method of claim 1, further comprising: after the post-treating step, washing the post-treated corneal extracellular matrix with a buffer and purified distilled water, followed by lyophilization for a predetermined period of time;
incising the lyophilized corneal extracellular matrix, followed by solubilization; and
adjusting the solubilized matrix to the pH suitable for the human body, and then inducing gelation of the matrix.

8. The method of claim 7, wherein in the solubilizing step, the corneal extracellular matrix is treated with one selected from the group consisting of protein hydrolases, corresponding to 0.01-0.05 mass percent of the corneal extracellular matrix, followed by addition of an acid treatment material and then stirring at 500-700 rpm for 44-52 hours.

9. The method of claim 7, further comprising: after the step of inducing gelation,
injecting the gelated bioink into a 3D printer, and preparing a cornea matching the size of a scanned affected part of a transplant recipient using the 3D printer;
transplanting the prepared cornea into the transplant recipient; and
allowing the transplanted cornea to grow in contact with cells in the periphery of the cornea of the transplant recipient.

10. A decellularization composition for a biocompatible tissue, the decellularization composition being a mixture for decellularization of a tissue provided from a tissue source and containing purified water and charcoal as active ingredients.

11. The decellularization composition of claim 10, wherein negative ions emitted from the charcoal stabilize negative charges on a surface of collagen or glycosaminoglycans (GAGs) constituting the extracellular matrix of the provided cornea.

12. The decellularization composition of claim 10, wherein the charcoal is porous, and adsorbs or removes impurities in the periphery of the provided cornea through pores thereof.

13. The decellularization composition of claim 10, wherein the mixture contains pulverized charcoal and a salt in the purified water.
